(19) United States
(12) Patent Application Publication
(10) Pub. No.: US 2022/0265878 A1
(43) Pub. Date: Aug. 25, 2022

(54) SMART, USER-FRIENDLY NETWORK CONNECTED DEVICES AND RELATED SYSTEMS AND METHODS FOR SANITIZING OR DISINFECTING ITEMS

(71) Applicant: Violux, Inc., Ladera Ranch, CA (US)

(72) Inventors: Sean Clottu, Ladera Ranch, CA (US); Andy Aoe, West Hills, CA (US); Matthew McRae, Laguna Niguel, CA (US)

(21) Appl. No.: 17/678,987

(22) Filed: Feb. 23, 2022

Related U.S. Application Data
(60) Provisional application No. 63/153,209, filed on Feb. 24, 2021.

(51) Int. Cl. A61L 2/10; A61L 2/24

(52) U.S. Cl. CPC A61L 2/10; A61L 2/24; A61L 2202/122; A61L 2202/14

(57) ABSTRACT

A sanitizing system is disclosed. In various embodiments, the sanitizing system includes a sanitizing apparatus, the sanitizing apparatus including an ultraviolent light source and a processor configured to control operation of the ultraviolet light source based on one or more user inputs received by the processor; and a remote device configured to transmit the one or more user inputs to the processor.

### CROSS-REFERENCE TO RELATED APPLICATIONS

This patent application claims the benefit of U.S. Provisional Application No. 63/153,209 filed Feb. 24, 2021, which application is incorporated by reference in its entirety.

### FIELD

The present disclosure relates generally to antimicrobial sterilization equipment and, more particularly, to systems and methods use to sterilize or disinfect items.

### BACKGROUND

Items benefitting from periodic sterilization or disinfection are ubiquitous, and may include, for example, toys, food, portable electronic devices, money, utensils and a variety of other items having complex shapes and sizes, comprised of various materials and surfaces, or nooks and crannies within components used to house devices. Current methods used to sterilize or disinfect such items, such as mild detergents may minimize any damage to the items, but do not adequately sterilize or disinfect the equipment. Thus, microorganisms may be shielded within air bubbles or under dirt, grease, oil or clumps of microorganisms. Furthermore, cellular proteins and other byproducts may reduce the efficacy of some liquid germicides. Similarly, any subsequent chemical treatments to sterilize or disinfect the items may cause damage either internal or external components of the items, particularly electronic items such as portable electronic devices. Disinfecting solvents such as benzenes or alcohols or alternative disinfectants may be used to clean some items, such as, for example, portable electronic devices or other electronic equipment following a pre-washing step. These chemicals have been known, however, to adversely impact or degrade plastic or polymerics materials used to fabricate housings or electronic components disposed within such housings. Further, nooks and crannies or crevices within the housings or other items of complex shapes create situations where the chemicals used to sterilize or disinfect may not reach and destroy all the microorganisms or pathogens residing on the items.

To achieve desirable and more complete sterilization and disinfection, the use of ultraviolet (UV) radiation has been investigated. UV-C radiation, having wavelengths in the range of 100-280 nanometers (nm), is effective at killing or destroying pathogens, germs or infectious microorganisms, such as, for example, viruses, bacteria, protozoa, prions, viroids or funguses. Electromagnetic radiation at UV-C wavelengths tends to corrupt the deoxyribonucleic acid (DNA) molecules of such pathogens and thereby prevents them from replicating. As a result, electromagnetic radiation at UV-C wavelengths is efficacious at preventing the aforementioned viruses, bacteria, protozoa, prions, viroids or funguses or other infectious microorganisms from proliferating as diseases.

### SUMMARY

A sanitizing system is disclosed. In various embodiments, the sanitizing system includes a sanitizing apparatus, the sanitizing apparatus including an ultraviolent light source and a processor configured to control operation of the ultraviolet light source based on one or more user inputs received by the processor; and a remote device configured to transmit the one or more user inputs to the processor.

In various embodiments, the remote device comprises a smartphone. In various embodiments, the remote device is a smart speaker. In various embodiments, the ultraviolet light source comprises an ultraviolent light emitting diode. In various embodiments, the ultraviolent light source is configured to operate at a wavelength within a range between 100 nm and 280 nm.

In various embodiments, the sanitizing apparatus includes a door and a first sensor configured to detect a door status. In various embodiments, a second sensor is configured to detect a current received by the ultraviolet light source. In various embodiments, a second sensor is configured to detect a voltage across the ultraviolet light source.

In various embodiments, the processor is configured to transmit a door status data report to the remote device. In various embodiments, the processor is configured to transmit a current signal representative of a current through the ultraviolet light source to the remote device. In various embodiments, the processor is configured to transmit a voltage signal representative of a voltage across the ultraviolet light source to the remote device. In various embodiments, the processor is configured to transmit an anomaly data report to the remote device. In various embodiments, the anomaly data report is representative of an anomalous current through the ultraviolet light source. In various embodiments, the anomaly data report is representative of an anomalous voltage across the ultraviolet light source.

In various embodiments, the system includes a database configured to store statistical data representative of operation of the sanitizing apparatus. In various embodiments, the database is a cloud-based database.

In various embodiments, the remote device is configured to receive data representative of operation of the sanitizing apparatus. In various embodiments, the data representative of operation of the sanitizing apparatus includes a use statistic or an error message. In various embodiments, the processor is configured to receive data representative of a user proximity to the sanitizing apparatus.

The foregoing features and elements may be combined in any combination, without exclusivity, unless expressly indicated herein otherwise. These features and elements as well as the operation of the disclosed embodiments will become more apparent in light of the following description and accompanying drawings.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the following detailed description and claims in connection with the following drawings. While the drawings illustrate various embodiments employing the principles described herein, the drawings do not limit the scope of the claims.

FIGS. 1A and 1B are schematic illustrations of a sanitizing apparatus, in accordance with various embodiments;

FIG. 2 is a schematic illustration of a sanitizing system, in accordance with various embodiments; and

FIG. 3 illustrates an operational flowchart of a sanitizing system, in accordance with various embodiments.

### DETAILED DESCRIPTION

The following detailed description of various embodiments herein makes reference to the accompanying drawings, which show various embodiments by way of illustration. While these various embodiments are described in sufficient detail to enable those skilled in the art to practice the disclosure, it should be understood that other embodiments may be realized and that changes may be made without departing from the scope of the disclosure. Thus, the detailed description herein is presented for purposes of illustration only and not of limitation. Furthermore, any reference to singular includes plural embodiments, and any reference to more than one component or step may include a singular embodiment or step. Also, any reference to attached, fixed, connected, or the like may include permanent, removable, temporary, partial, full or any other possible attachment option. Additionally, any reference to without contact (or similar phrases) may also include reduced contact or minimal contact. It should also be understood that unless specifically stated otherwise, references to “a,” “an” or “the” may include one or more than one and that reference to an item in the singular may also include the item in the plural. Further, all ranges may include upper and lower values and all ranges and ratio limits disclosed herein may be combined.

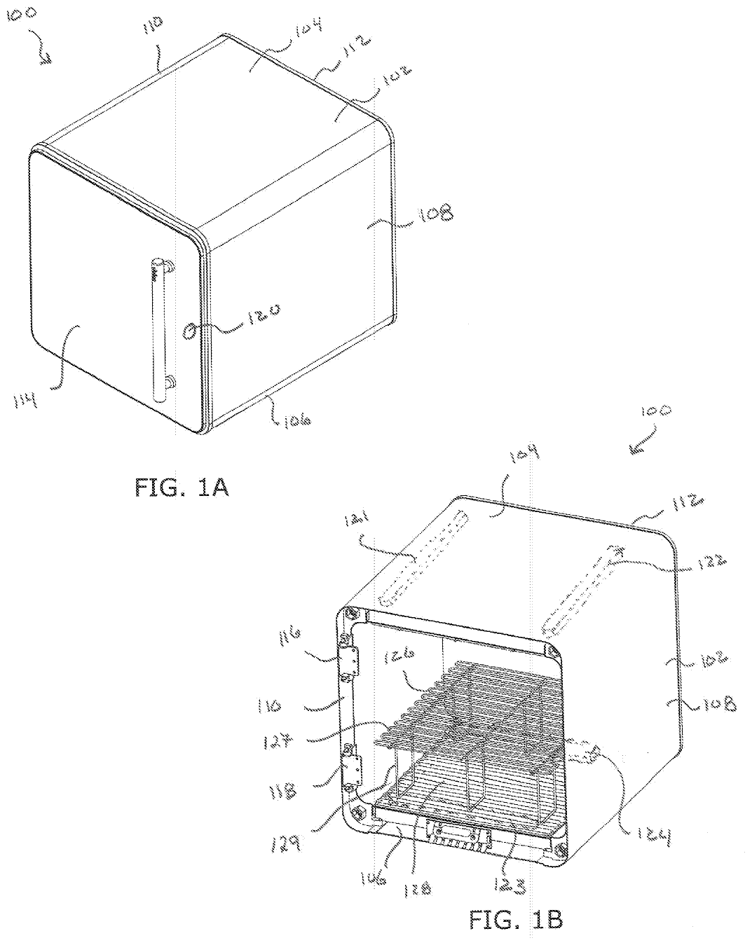

Referring now to the drawings, FIGS. 1A and 1B provide schematic views of a sanitizing apparatus 100, in accordance with various embodiments. The sanitizing apparatus 100 may include an enclosure 102 that defines an hollow interior bounded by a plurality of walls, including, for example, an upper wall 104, a lower wall 106, a first side wall 108, a second side wall 110 and a rear wall 112. A door 114, disposed opposite the rear wall 112 completes the enclosure 102. The door 114 is configured to open and thereby provide access to the hollow interior and to close and thereby enclose or envelope the hollow interior. Referring more specifically to FIG. 1B (illustrating the sanitizing apparatus 100 with the door 114 removed), a first hinge 116 and a second hinge 118 are mounted to the second side wall 110 and provide a hinged structure configured to open and close the door 114, as described above. As illustrated in FIG. 1A, in various embodiments, the door 114 may include a lock mechanism 120, which may include any suitable lock mechanism, including, for example, an unsecure lock mechanism, such as, for example, a push-button lock mechanism, exhibiting limited security, or a secure lock mechanism, such as, for example, a combination lock mechanism or a keyed-lock mechanism, exhibiting higher security to prevent unwanted or untimely opening of the enclosure—e.g., while the sanitizing apparatus 100 is performing a sanitizing operation.

Still referring primarily to FIG. 1B, the sanitizing apparatus 100 includes one or more emitters configured to provide a source of and to emit electromagnetic radiation. For example, and as illustrated, the sanitizing apparatus may include a plurality of emitters, including for example, a first emitter 121, a second emitter 122, a third emitter 123 and a fourth emitter 124. In more detail, the first emitter 121 and the second emitter 122 (illustrated using hidden lines) are mounted to an interior surface of the upper wall 104 and configured to direct electromagnetic energy downward and into the hollow interior of the enclosure 102. In various embodiments, the first emitter 121 and the second emitter 122 may be elongate devices, disposed to run longitudinally between the rear wall 112 and the door 114 (when closed). Similarly, the third emitter 123 and the fourth emitter 124 (illustrated using hidden lines) are mounted to an interior surface of the upper wall 110 and configured to direct electromagnetic energy upward and into the hollow interior of the enclosure 102. In various embodiments, the third emitter 123 and the fourth emitter 124 may also be elongate devices, disposed to run laterally between the first side wall 108 and the second side wall 110. In various embodiments, a tray structure 126 is disposed within the hollow interior of the enclosure 102 and configured to position an item or a plurality of items within the proximity of the plurality of emitters. The tray structure 126 may include, for example, a first tray 127 and a second tray 128, with the first tray 127 and the second tray 128 being spaced a distance from one another by one or more spacers 129. In various embodiments, the components comprising the tray structure 126 may be fabricated from materials—e.g., stainless steel or quartz glass—that are resistant to (or even transparent to) the electromagnetic radiation used in the sanitizing apparatus 100.

Referring specifically to the plurality of emitters illustrated in FIG. 1B, each emitter may be configured, in various embodiments, to emit electromagnetic radiation in the UV-C range of wavelengths (e.g., 100 nm-280 nm). While the disclosure focuses on this range of wavelengths, the disclosure contemplates use of other ranges of electromagnetic radiation, such as, for example: UV-A (315 nm-400 nm), which exhibits little effect on pathogens; UV-B (280 nm-315 nm), which exhibits some ability to affect damage against some pathogens; certain wavelengths of ultraviolent radiation (UV) or infrared radiation (IR); and certain wavelengths of ionizing or non-ionizing radiation. While the various spectra above identified, or combinations thereof) are contemplated, the disclosure focuses primarily on electromagnetic radiation in the UV-C range of wavelengths as providing the most efficacious wavelength for destroying the various viruses, bacteria, protozoa, prions, viroids or funguses or other infectious microorganisms referred to above, typically by destroying or adversely altering the DNA within the various pathogens or microorganisms. In various embodiments, each of the plurality of emitters described above may themselves comprise pluralities of smaller emitters, such as, for example, ultraviolet light emitting diodes (UVLEDs) that are tuned to emit electromagnetic radiation in the various ranges described above, including electromagnetic radiation in the UV-C range of wavelengths. Other sources of electromagnetic radiation contemplated by the disclosure include, without limitation, UV lamps, such as, for example, mercury, xenon, amalgam and excimer lamps.

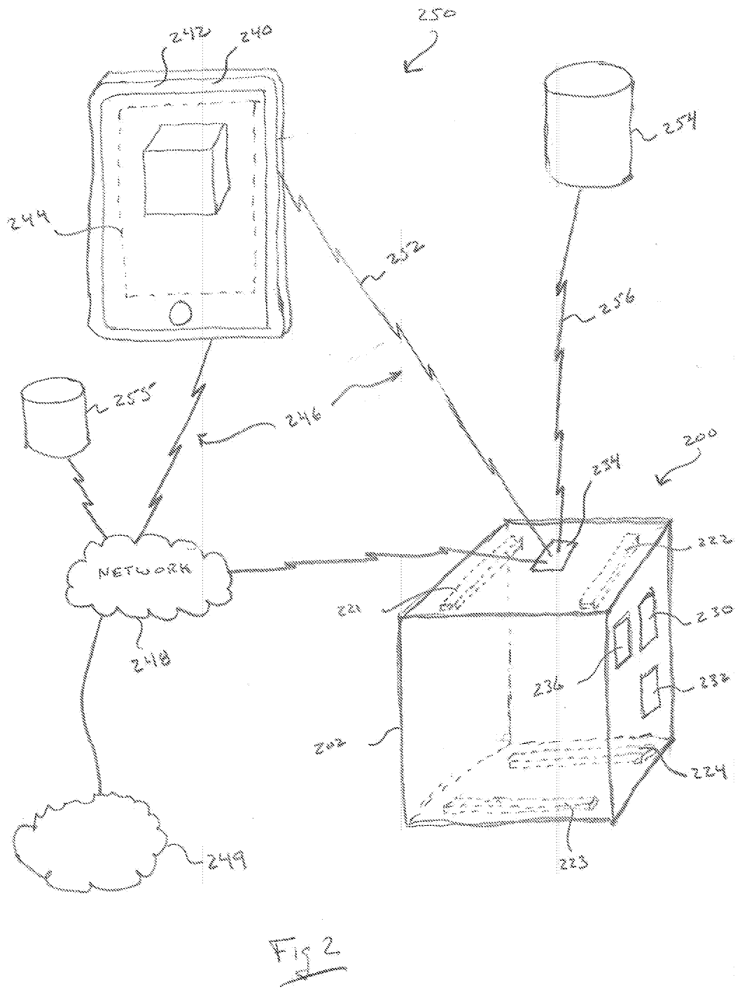

Referring now to FIG. 2, a sanitizing system 250 is illustrated, in accordance with various embodiments. The sanitizing system 250 includes a sanitizing apparatus 200, similar to the sanitizing apparatus 100 described above with reference to FIGS. 1A and 1B. The sanitizing apparatus 100 includes a plurality of emitters, including, for example, a first emitter 221, a second emitter 222, a third emitter 223 and a fourth emitter 224, each of which is housed within an enclosure 202. In addition to the various components described above with reference to FIGS. 1A and 1B, which are not repeated here, the sanitizing apparatus 200 includes a control module 230, a sensor module 232 and a communication interface 234. The control module 230 is configured to receive, for example, user commands (e.g., on/off commands, power density level commands for the plurality of emitters and time duration of operation commands) and sensor output signals from the sensor module 232. The user commands may be received via a manual input module 236 located on the enclosure 202 or via signals received at the communication interface 234, which signals may be initiated via a remote device 240, such as, for example, a smartphone, a mobile phone, an pad or a tablet, a computer or some other electronic device located remote from the sanitizing apparatus 200. Based on the user command signals and the sensor output signals, the control module controls operation of a sanitizing process performed by the sanitizing apparatus 200. As described further below, the sensor module 232 may include various sensors (e.g., a first sensor, a second sensor, etc.) configured to detect, for example, the status of the door (e.g., whether the door 114 is opened or closed) or the current being provided to an emitter or to each of the plurality of emitters.

In various embodiments, for example, the remote device 240 comprises a smartphone 242 that includes an application module 244 configured to process operational data concerning operation of the sanitizing apparatus 200. Signals concerning such operation are then received or transmitted, between the remote device 240 and the sanitizing apparatus 200, via a communication link 246. In various embodiments, the communication link 246 may comprise a network 248, such as, for example, an internet or a wireless communication system, typical of those used to receive and transmit phone calls, text messages or email communications between users. The network 248 permits operational control and monitoring of the sanitizing apparatus 200 by a user from and remote location. In various embodiments, the communication link 246 comprises a direct link 252, such as, for example, a Bluetooth® connection, between the remote device 240 and the sanitizing apparatus 200. The direct link 252 permits operational control and monitoring of the sanitizing apparatus 200 by a user from locations proximate the sanitizing apparatus 200 (e.g., from locations within a user's home or workplace). In various other embodiments, user input commands may be provided to the sanitizing apparatus 200 via other electronic devices, such as, for example, a smart speaker 254, which may also be configured to transmit and receive signals concerning operation and monitoring of the sanitizing apparatus 200 over a direct link 256, similar to the direct link 252 just described. In various embodiments, a remote smart speaker 255 may be connected to the network 248 and employed to control the sanitizing apparatus from remote locations in a manner similar to that of the remote device 240, which, in various embodiments, may be combined with the remote smart speaker 255. In various embodiments, such voice-based smart-speaker connections may optionally connect to and respond to commands from ecosystems such as, for example, Alexa, Google Home, Siri, Smart Things and other similar technologies to respond to events, voice commands and be included in smart home routines.

Additionally, in various embodiments, the sanitizing apparatus 200 may be configured to respond to geofencing events, such as, for example, being turned on or off in response to a user's physical location within or without a geofence, which may be considered a home or neighborhood perimeter. For example, a GPS-based application on the remote device 240 may generate physical coordinates of the remote device 240, with the physical coordinates alerting the remote device 240 to turn on or off the sanitizing apparatus 200 or the control module 230 turning on or off the device based on information it receives over the network 248 concerning the physical coordinates of the user.

In various embodiments, the sanitizing system 250 may be connected to, or include, a cloud-based database 249. In such embodiments, the sanitizing apparatus 200 may be configured to report various statistical information to a back end system to monitor various events, such as, for example, on/off events, durational periods of being on or off, elapsed or maximum dosage levels of electromagnetic radiation and the frequency levels of such dosage levels, emitter status or health, emitter malfunctions or other error states, either for a single sanitizing apparatus or for a plurality of sanitizing apparatus positioned at a plurality of locations. Where a plurality of sanitizing apparatus is at issue, the cloud-based database may facilitate an entity to rent or lease the plurality of sanitizing apparatus among a plurality of users and to collect and analyze various use statistics and error messages at a central location, rather than the location of each sanitizing apparatus. The database may also be employed by various of the users to monitor individual sainting apparatus to ensure employees are following required protocols—e.g., to monitor and ensure restaurant employees or healthcare workers are sanitizing various equipment at required intervals.

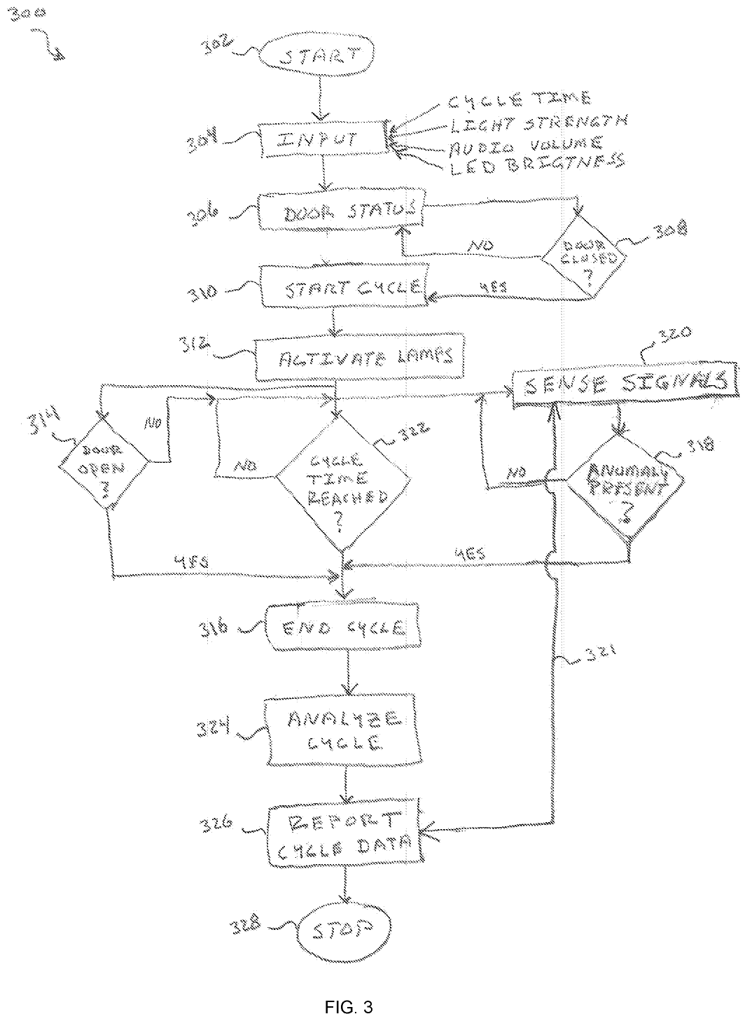

Referring now to FIG. 3, an operational flowchart 300 of a sanitizing system, such as, for example, the sanitizing system 250 described above with reference to FIG. 2, is provided. The operational flowchart 300 commences at a start step 302, which may represent the sanitizing system 250 being powered on. Powering on of the sanitizing system 250 may occur manually (e.g., via a push button on the sanitizing apparatus 200) or remotely (e.g., via one of the remote device 240 or the smart speaker 254). The operational flowchart 300 continues at an input step 304. The input step 304 includes receiving user inputs either manually (e.g., via a push button on the sanitizing apparatus 200) or remotely (e.g., via one of the remote device 240 or the smart speaker 254). The disclosure contemplates myriad inputs that may be received, including, without limitation, cycle time or duration, light strength or power, audio volume (e.g., for the smart speaker 254), LED brightness (e.g., the brightness of LED indicators on the sanitizing apparatus 200), identification of the item(s) being sanitized and any other inputs that may be useful for operation of the sanitizing system 250.

Following receipt of the user inputs, and prior to commencing a sanitizing operation, the sanitizing system 250 monitors status of the door (e.g., the door 114 described above with reference to FIG. 1A). For example, in concert with a door status step 305, the sanitizing system 250 monitors whether the door is open or closed at a first door status query step 308. Once the door is confirmed closed, the sanitizing system 250 so informs the user and proceeds to a start cycle step 310. At the start cycle step 310 of the operational flowchart 300, the user provides a start cycle input to commence the sanitizing operation. Similar to the user inputs described above, the start cycle input is provided manually (e.g., via a push button on the sanitizing apparatus 200) or remotely (e.g., via one of the remote device 240 or the smart speaker 254). Following receipt of the start cycle input, the sanitizing operation commences by powering or activating the UV lamps, which may comprise any of the UV light sources described above or even combinations thereof, at an activate lamps step 312.

Following activation of the UV lamps, several monitoring steps are commenced. The monitoring steps include, for example, a second door status query step 314, similar to the first door status query step 308 described above. As illustrated in the operational flowchart 300, if the door is determined to be opened during the sanitizing operation, the operation is terminated at an end cycle step 316. The second door status query step 314 is repeated at a suitable frequency, such that exposure to UV light is minimized following inadvertent opening of the door during a sanitizing operation. At the same time, an anomaly query step 318 is performed to detect surges or troughs in the electrical current being delivered to the UV lamps (or the voltage across the UV lamps). The query follows measurement of one or more signals at a signal measurement step 320. Various of the signals may include, for example, electrical current through the emitters, voltage across the emitters or brightness of the emitters, each of which may be sensed or measured via the sensor module 232 described above with reference to FIG. 2. As illustrated, in the event an anomaly is detected, the operation is terminated at the end cycle step 316. The anomaly query step 318 is also repeated at a suitable frequency, such that the sanitizing apparatus 200 does not experience damage (e.g., via a power surge) or provide an incomplete sanitization (e.g., via an underpowered UV lamp) during the cycle time (or duration) of the sanitizing operation. At the same time the second door status query step 314 and the anomaly query step 318 are being performed, a cycle time query step 322 is also being performed. The cycle time query step 322 monitors the elapsed time of the sanitizing operation and, once the elapsed time exceeds the cycle time input by the user, the sanitizing operation is terminated at the end cycle step 316.

Following termination of the sanitizing operation at the end cycle step 316, various cycle data is analyzed at an analyze cycle step 324 and then reported to the user a report cycle data step 326. In various embodiments, the cycle data includes, without limitation, information concerning termination of the sanitizing operation. For example, if the termination results from the door being opened prior to the cycle time being reached, such is reported to the user in the form of a door status data report (e.g., a report indicating the door was prematurely opened). Similarly, if the termination results from an anomaly being detected, information concerning the anomaly is analyzed and reported to the user in the form of an anomaly data report. The information may include, for example, whether the anomaly signals a failure of one or more of the UV lamps or a failure of the circuitry providing power to one or more of the UV lamps. In various embodiments, the report cycle data step 326 is carried out by providing the cycle data to the user via one or more of audio signals generated at the smart speaker 254 or audio or visual signals generated at the remote device 240. The cycle data may also be stored by the processor 230 within memory comprised within the processor 230 or at the remote device 240. Once the foregoing steps are complete, the operational flowchart 300 terminates at a stop step 328. In various embodiments, the signals being sensed at the signal measurement step 320 may be reported on the fly or at a predetermined frequency at the report cycle data step 326 via a link 321 that continually provides the signal data to a transmitter used to transmit the data to various of the above identified receivers or to the cloud-based database 249 described above.

As indicated above, the foregoing steps and queries are performed by a controller or a processor (e.g., the processor 230). In various embodiments, the controller or processor includes a tangible, non-transitory memory configured to implement digital or programmatic logic. In various embodiments, for example, the processor 230 comprises one or more of an application specific integrated circuit (ASIC), a digital signal processor (DSP), a field programmable gate array (FPGA), a general purpose processor or some other programmable logic device, discrete gate, transistor logic, or discrete hardware component, and the tangible, non-transitory memory is configured to store instructions that are implemented by the processor 230 for performing the various functions described above with reference to the operational flowchart 300.

Benefits, other advantages, and solutions to problems have been described herein with regard to specific embodiments. Furthermore, the connecting lines shown in the various figures contained herein are intended to represent exemplary functional relationships or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching is used throughout the figures to denote different parts but not necessarily to denote the same or different materials.

Systems, methods and apparatus are provided herein. In the detailed description herein, references to “one embodiment,” “an embodiment,” “various embodiments,” etc., indicate the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Numbers, percentages, or other values stated herein are intended to include that value, and also other values that are about or approximately equal to the stated value, as would be appreciated by one of ordinary skill in the art encompassed by various embodiments of the present disclosure. A stated value should therefore be interpreted broadly enough to encompass values that are at least close enough to the stated value to perform a desired function or achieve a desired result. The stated values include at least the variation to be expected in a suitable industrial process, and may include values that are within 10%, within 5%, within 1%, within 0.1%, or within 0.01% of a stated value. Additionally, the terms “substantially,” “about” or “approximately” as used herein represent an amount close to the stated amount that still performs a desired function or achieves a desired result. For example, the term “substantially,” “about” or “approximately” may refer to an amount that is within 10% of, within 5% of, within 1% of, within 0.1% of, and within 0.01% of a stated amount or value.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Finally, it should be understood that any of the above described concepts can be used alone or in combination with any or all of the other above described concepts. Although various embodiments have been disclosed and described, one of ordinary skill in this art would recognize that certain modifications would come within the scope of this disclosure. Accordingly, the description is not intended to be exhaustive or to limit the principles described or illustrated herein to any precise form. Many modifications and variations are possible in light of the above teaching.

What is claimed is:

1. A sanitizing system, comprising:
a sanitizing apparatus, the sanitizing apparatus including an ultraviolent light source and a processor configured to control operation of the ultraviolet light source based on one or more user inputs received by the processor; and
a remote device configured to transmit the one or more user inputs to the processor.

2. The sanitizing system of claim 1, wherein the remote device comprises a smartphone.

3. The sanitizing system of claim 1, wherein the remote device is a smart speaker.

4. The sanitizing system of claim 1, wherein the ultraviolet light source comprises an ultraviolent light emitting diode.

5. The sanitizing system of claim 1, wherein the ultraviolent light source is configured to operate at a wavelength within a range between 100 nm and 280 nm.

6. The sanitizing system of claim 1, further comprising a door and a first sensor configured to detect a door status.

7. The sanitizing system of claim 6, further comprising a second sensor configured to detect a current received by the ultraviolet light source.

8. The sanitizing system of claim 6, further comprising a second sensor configured to detect a voltage across the ultraviolet light source.

9. The sanitizing system of claim 1, wherein the processor is configured to transmit a door status data report to the remote device.

10. The sanitizing system of claim 9, wherein the processor is configured to transmit a current signal representative of a current through the ultraviolet light source to the remote device.

11. The sanitizing system of claim 10, wherein the processor is configured to transmit a voltage signal representative of a voltage across the ultraviolet light source to the remote device.

12. The sanitizing system of claim 11, wherein the processor is configured to transmit an anomaly data report to the remote device.

13. The sanitizing system of claim 12, wherein the anomaly data report is representative of an anomalous current through the ultraviolet light source.

14. The sanitizing system of claim 13, wherein the anomaly data report is representative of an anomalous voltage across the ultraviolet light source.

15. The sanitizing system of claim 1, further comprising a database configured to store statistical data representative of operation of the sanitizing apparatus.

16. The sanitizing system of claim 15, wherein the database is a cloud-based database.

17. The sanitizing system of claim 1, wherein the remote device is configured to receive data representative of operation of the sanitizing apparatus.

18. The sanitizing system of claim 17, wherein the data representative of operation of the sanitizing apparatus includes a use statistic or an error message.

19. The sanitizing system of claim 1, wherein the processor is configured to receive data representative of a user proximity to the sanitizing apparatus.
